(19) United States
(12) Patent Application Publication
(10) Pub. No.: US 2008/0188499 A1
(43) Pub. Date: Aug. 7, 2008

(54) Carbo- and Hetero-Cyclic Antibiotics and Use Thereof

(75) Inventors: Suzanne Chamberland, Eastman (CA); Francois Malouin, Eastman (CA)

Correspondence Address: KLARQUIST SPARKMAN, LLP, 121 SW SALMON STREET, SUITE 1600, PORTLAND, OR, 97204, US

(73) Assignee: ULYSSES PHARMACEUTICAL PRODUCTS INC., Sherbrooke, QC (CA)

(21) Appl. No.: 11/575,862

(22) PCT Filed: Sep. 22, 2005
(86) PCT No.: PCT/CA05/01436

§ 371 Date: Nov. 7, 2007

Related U.S. Application Data
(60) Provisional application No. 60/612,148, filed on Sep. 23, 2004.

(51) Int. Cl. A61K 31/517; C07D 239/94; A01N 43/54; A61P 31/04; A01P 1/00

(52) U.S. Cl. 514/266.4; 544/293

(57) ABSTRACT

The present invention relates to compounds of the formula (1.0); and pharmaceutically acceptable salts of such compounds. The present invention relates to chemical entities containing a nitrofuran or other antibiotic linked to an activity enhancing distal ring system either directly or via an imine group, the vinyl group, the carbo- or hetero-cyclic chain or ring or a combination of an imine group or a vinyl group and a carbo- or hetero-cyclic chain or ring. Antibiotic activity is obtained, for example, by the nitrofuran moiety, while the remaining structure of the molecule contributes to additional antimicrobial activity and/or extends the antimicrobial spectrum of activity, by facilitating nitroreduction by microorganisms, uptake in target bacterial, and/or intracellular penetration, while also contributing to pharmacological properties (absorption, body distribution, and others).

### CROSS-REFERENCE TO RELATED APPLICATIONS

This application claims the benefit of U.S. Provisional Patent Application No. 60/612,148 filed Sep. 23, 2004, which is incorporated herein by reference.

### FIELD OF THE INVENTION

This invention relates to novel antibiotics and their use for the treatment or prophylaxis of microbial infections, or as antiseptics, sterilizants or disinfectants. These compounds exhibit an extended antimicrobial spectrum of activity and reduced undesired toxic side effects, as well as antibiotic activity against a wide spectrum of microorganisms, including organisms which are resistant to multiple antibiotic families.

### BACKGROUND OF THE INVENTION

The following review of the background of the invention is merely provided to aid in the understanding of the present invention and neither it nor any of the references cited within it are admitted to be prior art to the present invention.

Management of nosocomial or community-acquired bacterial infections is becoming increasingly difficult due to the emergence of bacteria resistant to one or multiple families of antibiotics. Unfortunately, the widespread and indiscriminant use of antibiotics has led to a rapid increase in the number of bacterial strains which are resistant to antibiotics. Most importantly, resistance has emerged among clinically important microorganisms which threaten the utility of the currently available arsenal of antibiotics. A global trend of increasing resistance to antibiotics, with wide variations according to geographical areas, is well documented by the World Health Organization and in the scientific literature.

There is a need for novel and effective antibiotics that are particularly active against microorganisms which are resistant to currently available drugs. For example, resistance of bacteria causing urinary tract infections to trimethoprimsulfamethoxazole, β-lactams and fluoroquinolones is becoming a major factor in the management of such infections. Despite the use of nitrofuran antibiotics for several decades, mainly for the treatment of urinary tract infections, resistance to agents of this family has remained low (0-2%) in commonly encountered microorganisms (Gupta K., Addressing antibiotic resistance. Dis Mon. 2003 February; 49(2):99-110; Nicolle L E, Urinary Tract Infection: Traditional pharmacologic therapies. 2003 February; 49(2):111-128). Nitrofurans have also been shown to be useful in the treatment of severe infections caused by multiresistant microorganisms.

However, there are only a few nitrofuran antibiotics currently used in humans for the treatment of infectious diseases and one is known by the generic name nitrofurantoin (commercial names include: Macrobid, Macrodantin, Furadantin). It is used in adults and children to treat acute urinary tract infections and to prevent recurrent urinary tract infections. A drawback of nitrofurantoin is that it does not have good potency (i.e., relatively high amounts are required to exert its antibacterial activity) and it does not have a wide spectrum of antimicrobial activity, which limits the use of this compound in treating bacterial infections. Besides, U.S. Pat. Nos. 3,970,648, 3,973,021 and 3,974,277 disclose nitrofuran antibiotics of the following formulae: 2-[2-(5-nitro-2-furyl)vinyl]-4-(anilino)-quinazoline, 2-[2-(5-nitro-2-furyl)vinyl]-4-(p-hydroxy-anilino)-quinazoline, 2-[2-(5-nitro-2-furyl)vinyl]-4-(o-hydroxyanilino)-quinazoline, and 2-[2-(5-nitro-2-furyl)vinyl]-4-(m-hydroxyanilino)-quinazoline. These patents teach the use of these compounds as pesticides and animal growth promotants for improving feed efficiency in animals such as poultry, swine and cattle. Although these molecules gained the property of being adequate edible feed additives for animal growth promotion compared to quinazoline molecules having the nitrofuran group directly attached to it (U.S. Pat. No. 3,542,784), a drawback of the compounds from the above patents (U.S. Pat. Nos. 3,970,648, 3,973,021 and 3,974,277) is that the patents teach that they are now devoid of activity against important pathogens such as Escherichia coli, Staphylococcus aureus and Salmonella. It would be desirable to obtain nitrofurans which provide significant improvement of potency and expand the antimicrobial spectrum of activity. This means that lower amounts of compounds are required for in vitro and in vivo (in animals) antimicrobial action against a wider variety of pathogens affecting animals and humans.

Novel antibiotics with superior antimicrobial potency and improved pharmacological properties would provide an alternative for the treatment of severe infections caused by antibiotic-susceptible and antibiotic-resistant microorganisms.

### SUMMARY OF THE INVENTION

The present invention relates to chemical entities containing a nitrofuran or other antibiotic linked to an activity enhancing distal ring system either directly or via an imine group, vinyl group, carbo- or hetero-cyclic chain or ring or a combination of an imine group or a vinyl group and a carbo- or hetero-cyclic chain or ring. Antibiotic activity is obtained, for example, by the nitrofuran moiety, while the remaining structure of the molecule contributes to additional antimicrobial activity and/or extends the antimicrobial spectrum of activity, by facilitating nitroreduction by microorganisms, uptake in target bacteria, and/or intracellular penetration, while also contributing to pharmacological properties (absorption, body distribution, and others). The invention is described with reference to nitrofuran as the antibiotic moiety, however it will be understood that reference throughout to nitrofurans represents any moiety having antibiotic activity.

When present, the carbo or hetero-cyclic chain or ring (herein referred to as the “central structure”) possesses a dual role of enhancing the activity of the antibiotic such as nitrofuran and serving as a point of attachment for the distal ring system. The central structure can be a monocyclic ring of 3 to 8 atoms, preferentially a pyrazine or a triazine as well as a bicyclic system composed of 4 to 14 atoms, preferentially a quinazoline. The distal ring system is attached to the central structure along with the nitrofuran or other antibiotic functional group at the optimum positions for activity.

The distal ring system is either a hydroxyphenyl, catechol, biscatechol, triscatechol (the two former being held together by an appropriate scaffold), thiazole, thiazoline, oxazole, oxazoline, imidazole or imidazoline. When the distal ring system is thiazole, thiazoline, oxazole, oxazoline, imidazole or imidazoline, the 5 position of the distal ring system is attached to the central structure at the optimum positions for activity, while a substituted or unsubstituted phenyl or pyridine or other combinations of carbo- or hetero-cyclic structures with 3 to 8 atoms aliphatic or aromatic in nature, is attached at the 2 position. The distal ring system can also be a substituted or unsubstituted mono or bi carbo- or hetero-cyclic structure with 4 to 14 atoms which are aliphatic or aromatic in nature and any combinations with a substituted or unsubstituted mono or bi carbo- or hetero-cyclic aliphatic or aromatic structure having 3 to 8 atoms. In some cases, the distal ring system can also be replaced by an open chain structure containing one or more functional groups like hydroxamic acid that contributes to the antimicrobial activity or spectrum of activity. The term “distal ring system” as used herein encompasses such open chain structures.

In the distal ring system, the 2-phenyl oxazoline, oxazole, thiazoline, thiazole, imidazoline, imidazole or catechol are structurally related to biologically active microbial components such as mycobactins or tosiderophores used by Pseudomonas, Burkholderia and other bacteria as well as to inhibitors of bacterial lipid A biosynthesis. The present invention takes advantage of the properties of these ring structures to enhance and extend the antibacterial and pharmacological properties of nitrofurans.

The invention also includes pharmaceutically acceptable formulations of said compounds which exhibit antibiotic activity against a wide spectrum of microorganisms including organisms which are ordinarily not susceptible to nitrofuran and organisms that are resistant to multiple antibiotic families. These novel compounds are useful as antibacterial agents for treatment or prophylaxis of bacterial infections, or as antiseptics, or agents for sterilization or disinfection.

This invention includes compounds of the formula (1.0)

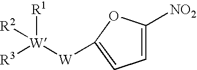

wherein
W is absent, vinyl (cis or trans —CH═CH—, preferably trans) or —N═CH—;
W′ is absent, or, shown with R¹, R² and R³, is

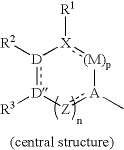

wherein D, D″, X, M, A and Z are each independently selected from CH, C, O, S, NH and N, however at least one of D or X is C, and preferably no more than two D, X, M, A or Z are O, S, NH or N, and D″ must be C or CH, unless R² and R³ are taken together to form a ring; the dotted line is an optional bond (no ring structure); and p and n are each independently selected from 0, 1, and 2;
R¹, R² and R³ are each independently selected from absent, hydrogen, halogen (includes halogen atoms fluorine, chlorine, bromine and iodine), CH₃, C₁-C₁₀ alkyl, C₂-C₁₀ alkenyl, C₂-C₁₀ alkynyl, C₁-C₁₀ alkoxy, C₂-C₁₀ alkenyloxy, C₂-C₁₀ alkynyloxy, aryl, OH, trifluoromethyl, methylenedioxy, phenoxy, OR⁴, CO₂R⁴, SO₂R⁴, pO (OR⁴)₂, CON(R⁴)₂, OAr, NH₂, NHR₄, NR₄, N(R⁴)₂, NHAr, SH, SR⁴, SAr, ═O, hydroxamic acid, heterocyclic ring, a solubilizing group as defined below, and a VQT moiety (the distal ring system) as defined below, wherein the alkyl, alkenyl, alkynyl, alkoxy, alkenyloxy or alkynyloxy group may be unsubstituted or substituted, preferably, with 1-5 halogen atoms or 1-2 OR⁴ groups, and aryl is, preferably, selected from the group consisting of: phenyl, naphthyl, indolyl, biphenyl, phenoxyphenyl, pyridyl, furanyl, thiophenyl and bithienyl, said aryl group being optionally substituted, preferably, by 1-3 groups selected from R⁴, a solubilizing group as defined below, and a VQT moiety as defined below; the solubilizing group can be, without limitation, preferably

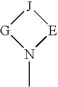

wherein G and E are each independently selected from CH₂, CH₂CH₂ and CH₂CH-alkyl; and J is O, NH or NCH₃; R⁴ is selected from CH₃, C₁-C₁₀ alkyl, C₂-C₁₀ alkenyl, C₂-C₁₀ alkynyl, C₁-C₁₀ alkoxy, C₂-C₁₀ alkenyloxy, C₂-C₁₀ alkynyloxy, aryl, heterocyclic ring, a solubilizing group as defined above, and aryl is, preferably, selected from the group consisting of: phenyl, naphthyl, indolyl, biphenyl, a substituted or unsubstituted mono or bi carbo- or hetero-cyclic structure having 4-14 atoms which are aliphatic or aromatic in nature and any combinations with a substituted or unsubstituted mono or bi carbo- or hetero-cyclic aliphatic or aromatic structure having 3 to 8 atoms;

### V is C, CH, N, NH or O;

Q acts as a stable or labile linker, for example selected from absent, C₁-C₁₀ alkyl, C₂-C₁₀ alkenyl, C₂-C₁₀ alkynyl, C₁-C₁₀ amine, C₁-C₁₀ alkoxy, C₂-C₁₀ alkenyloxy, C₂-C₁₀ alkynyloxy, phenyl, heterocyclic ring, —C(═O)—, —SO₂—, —PO(OT)₂-, —NOH, —(CH₂)ₜ—C(═O)—, and —(CH₂)ₜ—NH—C(═O)— wherein t is 0 to 10; and

### T is

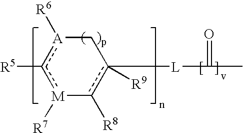

wherein A and M are independently selected from C, CH, O, NH, N and S; p, n and v are each independently selected from 0 and 1; and the dotted line represents optional additional bonds; and

R⁵, R⁶, R⁷, R⁸ and R⁹ are each independently selected from absent or as defined for R¹;

the preferred R⁵ is:

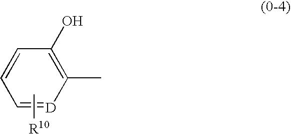

wherein D is independently selected from CH, C, O, S, NH and N;

and wherein R¹ is absent or as defined for R¹;

L is acting as a stable or labile linker, for example selected from absent, C₁ to C₁₀ alkyl, C₂ to C₁₀ alkenyl, C₂ to C₁₀ alkynyl, C₁-C₁₀ amine, C₁-C₁₀ alkoxy, C₂-C₁₁ alkenyloxy, C₂-C₁₀ alkynyloxy, phenyl, —C(═O)—, —PO(OT)₂-, —NOH—, —(CH₂)ₜ—C(═O)—, and —(CH₂)ₜ—NH—C(═O)— wherein t is 0 to 10, and a heterocyclic ring, wherein the heterocyclic ring is preferably

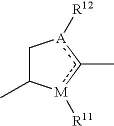

wherein A and M are independently selected from C, CH, N, NH, O, or S; and wherein R¹¹ and R¹² are independently selected from absent, or as defined for R¹; and wherein the dotted line represents optional additional bonds; R² and R³, when taken together in formula 1.0, (wherein W′ is the central structure), form in combination with the D and D″ atoms of the central structure to which they are fused, the following:

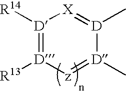

wherein X, D, D′, D″, D′″, Z are each independently selected from CH, C, O, S, NH, and N; and n is selected from 0, 1 and 2;

wherein R¹³ and R¹⁴ are each independently as defined for R¹;

and when R² and R³ are taken together in formula 1.0,

R¹ is as defined above, or

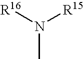

wherein R¹⁵ and R¹⁶ are each independently as defined for R⁴;

with the proviso that there must be present in the compound of formula 1.0 at one, two or three positions at least one VQT moiety which is selected from one or more of:

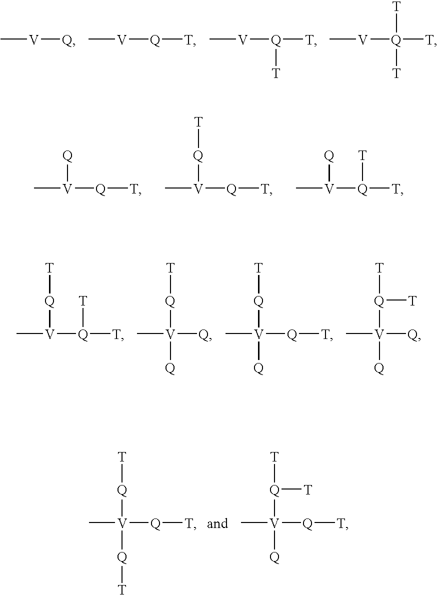

or enantiomers thereof;

wherein the compound of formula 1.0 has from 1 to 9 T, and each V and Q and T is selected independently;

and pharmaceutically acceptable salts of such compounds.

This invention also includes compounds similar to formula 1.0 wherein the nitrofuran moiety is replaced by another antibiotic moiety. Such another antibiotic moiety would be linked to the remainder of compound 1.0 through W.

Examples of preferred T are as follows:

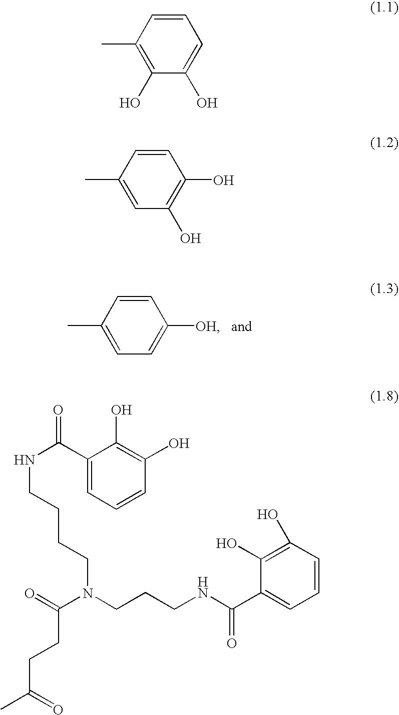

More preferrably, T is selected from

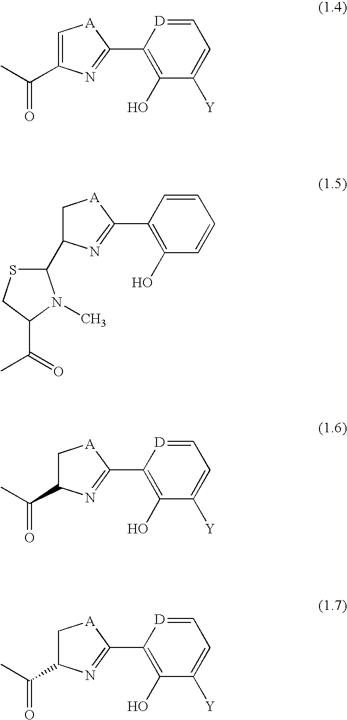

wherein A is selected from O, NH, NR and S; D is CH or N; and Y is absent or OH.

### BRIEF DESCRIPTION OF THE DRAWINGS

FIG. 1 graphs the incorporation of radiolabeled precursors into macromolecules in the presence of 2-[2-(5-nitro-2-furyl)vinyl]-4-(3,4-dihydroxyanilino)quinazoline (the compound VI of Example 1).

### DETAILED DESCRIPTION OF THE INVENTION

The present invention includes novel catecholpyrazine analogs of the formula

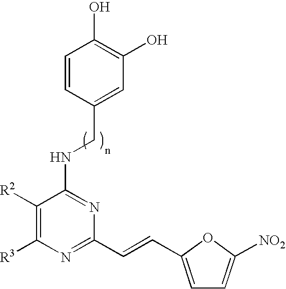

wherein R² and R³ are hydrogen, alkyl, aryl, OH, OR, OAr, NH₂, NHR, NHAr, SH, SR, or SAr, and n=0, 1, or 2.

The catecholquinazolines and other catechol-heterobicyclic analogs have the following formula:

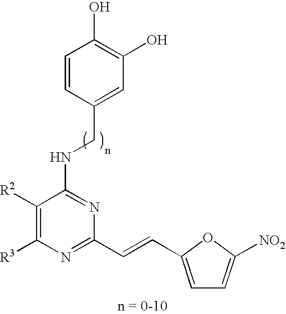

wherein R² and R³ are taken together as defined above. The catecholtriazene analogs have the following structure:

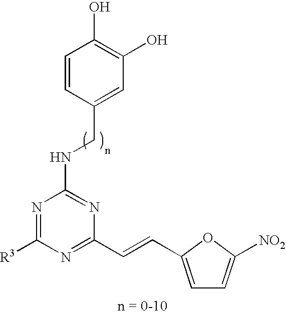

wherein R³ is hydrogen, alkyl, aryl, OH, OR, OAr, NH₂, NHR, NHAr, SH, SR, or SAr.
The biscatechol containing nitrofuran derivatives have the following structure:

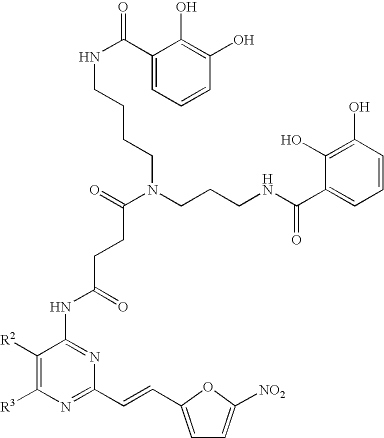

wherein R² and R³ can be taken together as defined above.
Derivatives based on pyrazine have the following structure:

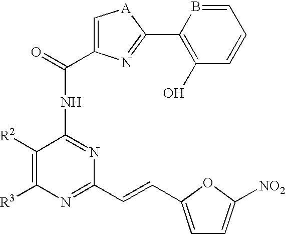

wherein A is O, NH, NR, S; R² and R³ are hydrogen, alkyl, aryl, OH, OR, OAr, NH₂, NHR, NHAr, SH, SR, or SAr, and B is CH or N.
Derivatives based on quinazoline have the following structure:

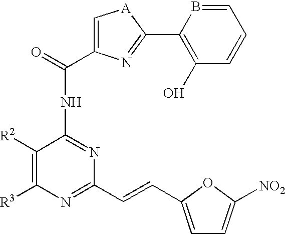

wherein A is O, NH, NR, or S; B is CH or N, and R² and R³ are taken together as defined above.

Derivatives based on hydroxyarylthiazolines, hydroxyaryloxazolines and hydroxyarylimidazolines have the following structures:

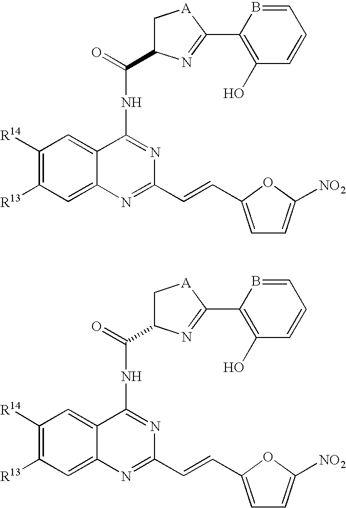

wherein A is O, NH, NR or S, R¹³ and R¹⁴ are hydrogen, alkyl, aryl, OH, OR, OAr, NH₂, NHR, NHAr, SH, SR, or SAr; and B is CH or N.

Compounds of the present invention can generally be made using the following general methods. Hydrochloric acid is reacted with anthranilamide and methanol to form anthranilamide hydrochloride. To this is added, in steps, hydrochloric acid, acetic anhydride and aqueous ammonia, forming 2-methyl-4-(3H)quinazolinone. Next 5-nitro-2-furancarboxaldehyde is added with acetic anhydride and sulfuric acid to form 2-[2-(5-nitro-2-furyl)vinyl]-4-(3H)quinazolinone, which is used to prepare chloro and anilino derivatives. For example, phosphorus pentachloride and phosphorus oxychloride were added to form 2-[2-(5-nitro-2-furyl)vinyl]-4-chloroquinazoline to which various functional groups can be added at the 4 position on the quinazoline. We refer to the Examples for a more detailed description of these methods.

In vitro and in vivo (in animals) tests have revealed the unique antimicrobial properties of the compound 2-[2-(5-nitro-2-furyl)vinyl]-4-(3,4-dihydroxyanilino) quinazoline and derivatives, and demonstrated that the spectrum of activity of these molecules is highly suitable for treatment of difficult-to-treat human infections. In particular, 2-[2-(5-nitro-2-furyl)vinyl]-4-(3,4-dihydroxy-anilino)quinazoline demonstrates activity against multiple Gram positive and Gram negative bacteria. Such a property is comparable to commercial drugs of the macrolide, β-lactam, or fluoroquinolone class. Moreover, 2-[2-(5-nitro-2-furyl)vinyl]-4-(3,4-dihydroxyanilino)quinazoline, being of a different structural class, is not affected by commonly found microbial mechanisms of resistance that have been developed over the recent years against most antimicrobial agents currently used clinically. Also, applicant has demonstrated that 2-[2-(5-nitro-2-furyl)vinyl]-4-(3,4-dihydroxyanilino)-quinazoline, administrated by gavage, is active in vivo in a mouse model of infection, thus indicating oral bioavailability and relatively low toxicity. Initial mode of action studies also demonstrated that the antibiotic effect of 2-[2-(5-nitro-2-furyl)vinyl]-4-(3,4-dihydroxyanilino)quinazoline is produced through an inhibition of DNA metabolism, an essential cell process for microbes. All these antimicrobial and chemical properties represent those of a potent and safe antibiotic molecule. Certain terms in this application are described in the following text.

The term “alkyl” refers to the radical of saturated aliphatic groups including straight chain alkyl groups, branched-chain alkyl groups, cycloalkyl (alicyclic) groups, alkyl substituted cycloalkyl groups, and cycloalkyl substituted alkyl groups. Typical alkyl groups include, but are not limited to, methyl, ethyl, propyl, isopropyl, butyl, isobutyl, t-butyl, pentyl, isopentyl, hexyl, etc. The alkyl groups can be (C₁-C₁₀) alkyl, more preferably (C₁-C₆) alkyl and even more preferably (C₂-C₄) alkyl.

The term “alkyl” can encompass a “substituted alkyl” having substituents replacing a hydrogen on one or more carbons of the hydrocarbon backbone. Such substituents can include, for example, halogen, hydroxyl, carbonyl (such as carboxyl, ketones (including alkylcarbonyl and arylcarbonyl groups), and esters (including alkyloxycarbonyl and aryloxycarbonyl groups)), thiocarbonyl, acyloxy, alkoxyl, phosphoryl, phosphonate, phosphinate, amino, acylamino, amido, amidine, imino, cyano, nitro, azido, sulfhydryl, alkylthio, sulfate, sulfonate, sulfamoyl, sulfonamido, heterocyclyl, aralkyl, or an aromatic or heteroaromatic moiety. The moieties substituted on the hydrocarbon chain can themselves be substituted, if appropriate. For instance, the substituents of a substituted alkyl may include substituted and unsubstituted forms of aminos, azidos, iminos, amidos, phosphoryls (including phosphonates and phosphinates), sulfonyls (including sulfates, sulfonamidos, sulfamoyls and sulfonates), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The terms “alkenyl” and “alkynyl” refer to unsaturated aliphatic groups analogous in length and possible substitution to the alkyls described above, but that contain at least one double or triple bond respectively. An “alkenyl” is an unsaturated branched, straight chain, or cyclic hydrocarbon radical with at least one carbon-carbon double bond. The radical can be in either the cis or trans conformation about the double bond(s).

Typical alkenyl groups include, but are not limited to, ethenyl, propenyl, isopropenyl, butenyl, isobutenyl, tert-butenyl, pentenyl, hexenyl, etc. An “alkynyl” is an unsaturated branched, straight chain, or cyclic hydrocarbon radical with at least one carbon-carbon triple bond. Typical alkynyl groups include, but are not limited to, ethynyl, propynyl, butynyl, isobutynyl, pentynyl, hexynyl, etc.

The term “halogen” refers to fluoro, chloro, bromo or iodo or fluoride, chloride, bromide or iodide or fluorine, chlorine, bromine or iodine.

The term “amine” refers to an organic compound containing nitrogen, having (C₁-C₁₀), more preferably (C₁-C₆) and even more preferably (C₂-C₄) carbon atoms, this compound may further bear one or more substituents as set forth above in the definition of alkyl.

The term “alkoxy” refers to a straight chain saturated hydrocarbon or branched saturated hydrocarbon bonded through an oxy. Examples of alkoxy include (C₁-C₁₀)alkoxy, more preferably (C₁-C₆)alkoxy and even more preferably (C₂-C₄) alkoxy. Included in the definition of the term “alkoxy” are those alkoxy further bearing one or more substituents as set forth above in the definition of alkyl.

The terms “alkenyloxy” and “alkynyloxy” refer to organic compounds analogous in length and possible substitution to alkoxy described above, but that contain at least one double or triple bond respectively.

The term “aryl” refers to aromatic radicals having 3-14 ring atoms and at least one ring having a conjugated pi electron system and encompasses “heteroaryl” compounds. Preferably at least two, more preferably at least four, of the ring atoms are carbon atoms. The term “heteroaryl” refers to an aromatic heterocyclic group usually with one or more, preferably no more than two, heteroatoms selected from O, S and N in the ring and which aryl and heteroaryl are analogous in possible substitution to the alkyls described above.

The term “heterocyclic ring” refers to a ring structure which can be saturated, unsaturated or aromatic, having 3-14 ring atoms, with one or more, preferably no more than two, heteroatoms selected from O, S and N in the ring, and the ring may further bear one or more substituents as set forth above in the definition of alkyl. Preferably at least two, more preferably at least four, of the ring atoms are carbon atoms.

The term “solubilizing group” refers to any group that improves the water solubility of the compound. Such a group can include, without limitation, the following

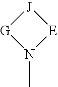

wherein G and E are each independently selected from CH₂, CH₂CH₂ and CH₂CH-alkyl, and J is O, NH or NCH₃.

In various embodiments, the nitrofurans of the present invention may be used therapeutically in formulations or medicaments to prevent or treat bacterial infections. The invention provides corresponding methods of medical treatment, in which a therapeutic dose of a nitrofuran of the present invention is administered in a pharmacologically acceptable formulation, e.g. to a patient or subject in need thereof. Accordingly, the invention also provides therapeutic compositions comprising a nitrofuran of the present invention, and a pharmacologically acceptable diluent, adjuvant, excipient or carrier. In one embodiment, such compositions include a nitrofuran of the present invention in a therapeutically or prophylactically effective amount sufficient to treat or prevent a bacterial infection. The therapeutic composition may be soluble in an aqueous solution at a physiologically acceptable pH.

A “therapeutically effective amount” refers to an amount effective, at dosages and for periods of time necessary, to achieve the desired therapeutic result, such as a reduction of bacterial infection. A therapeutically effective amount of a nitrofuran of the present invention may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting the rate of bacterial infection-related disease onset or progression. A prophylactically effective amount can be determined as described above for the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, micro-emulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, a nitrofuran of the present invention can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Therapeutic compositions formulated as liposomes or other ordered structure can be prepared with, for example, antibodies to help delivery of a nitrofuran of the present invention to specific microbes, cells, tissues or organs. Many methods for the preparation of such formulations are patented or generally known to those skilled in the art.

Sterile injectable solutions can be prepared by incorporating the active compound (e.g. a nitrofuran of the present invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, a nitrofuran of the present invention may be formulated with one or more additional compounds that enhance the solubility of the nitrofuran.

In accordance with another aspect of the invention, therapeutic compositions of the present invention, comprising a nitrofuran of the present invention, may be provided in containers or commercial packages which further comprise instructions for use of the nitrofuran for the prevention and/or treatment of bacterial infection.

Accordingly, the invention further provides a commercial package comprising a nitrofuran of the present invention, or the above-mentioned therapeutic composition, together with instructions for the prevention and/or treatment of bacterial infection.

The invention further provides a use of a nitrofuran of the present invention for prevention and/or treatment of bacterial infection. The invention further provides a use of a nitrofuran of the present invention for the preparation of a medicament for prevention and/or treatment of bacterial infection.

The invention further provides a use of a nitrofuran of the present invention as an antiseptic, sterilizant, or disinfectant.

All patents, patent applications and publications mentioned herein, both supra and infra, are hereby incorporated by reference.

While the invention has been described with reference to certain specific embodiments and will be described in the following Examples, it is understood that it is not to be so limited since alterations and changes may be made therein which are within the full and intended scope of the appended claims.

Now in order to more particularly define some embodiments of the present invention, the following Examples provide details of specific compounds of the invention, methods of producing the same and results from testing such compounds.

### EXAMPLE I

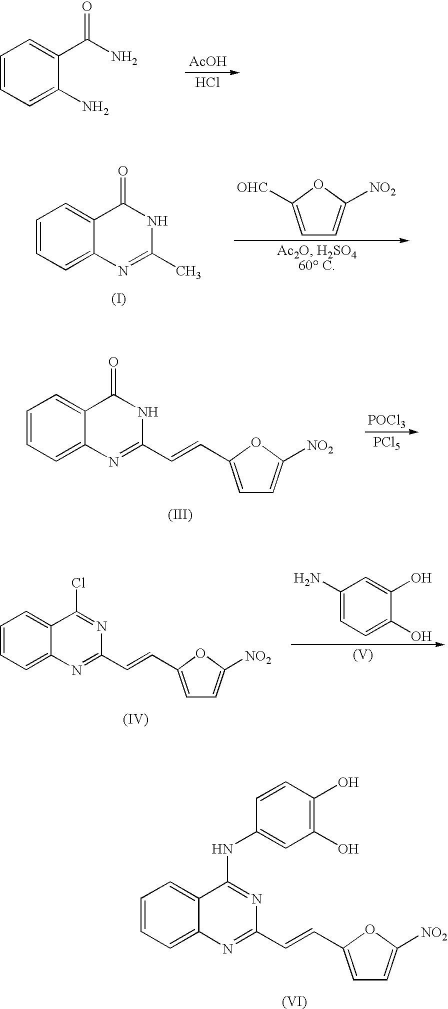

### 2-methyl-4-(3H)quinazolinone (I)

Anthranilamide hydrochloride was prepared by adding 20 ml of concentrated hydrochloric acid (37% by weight) to a solution of 27.3 g of anthranilamide in 200 ml of methanol.

This mixture was cooled in an ice bath to precipitate the hydrochloride which was then collected and dried to obtain a product. A 17.4 g (0.1 mole) portion of the hydrochloride thus obtained was refluxed for 3 hours with 100 ml acetic anhydride and allowed to stand overnight. The mixture was then cooled in an ice bath and the solids collected by filtration on a Buchner funnel. The filter cake was slurried in 100 ml of water, and warmed to aid solution and then 28% aqueous ammonia was added until the mixture was alkaline. After cooling, the 2-methyl-4-(3H)quinazolinone precipitated as a solid, was then collected, washed with a small amount of cold water and dried at 70° C. to obtain 6.72 g of the desired product.

### 5-nitro-2-furancarboxaldehyde (II)

A total of 86.5 g of 5-nitrofurfurylidine diacetate was added in small portions to 90 ml of sulfuric acid (73% by weight) over a period of 10 to 15 min. The mixture was stirred for 30 min at ambient temperature, 10 min at 50° C., cooled to 30° C., and then poured onto 150 g of crushed ice. The mixture was filtered, sucked as dry as possible on a Buchner funnel with the aid of a rubber dental dam and this afforded 51.5 g of 5-nitro-2-furancarboxaldehyde which melted at 32°-34° C. 2-[2-(5-nitro-2-furyl)vinyl]-4-(3H)quinazolinone (III)

To 16 g (0.1 mole) 6-fluoro-2-methyl-4-(3H)quinazolinone were added 100 ml acetic anhydride, 0.5 ml 96% sulfuric acid and 20 g (0.14 mole) 5-nitro-2-furancarboxaldehyde and the mixture was stirred 2 hours at 50° C.-60° C. The reaction mixture was poured into water and boiled 10 min. After it stood overnight, the product was collected by filtration, washed with water, then methanol. A yellow solid was obtained. This solid 2-[2-(5-nitro-2-furyl)vinyl]-4-(3H)quinazolinone was used to prepare the chloro (IV) and anilino (V) derivatives described below.

### 2-[2-(5-nitro-2-furyl)vinyl]-4-chloroquinazoline (IV)

A 500 ml 3 necked flask fitted with a stirrer, reflux condenser and protected by a calcium chloride trap was charged with 9.0 g of phosphorus pentachloride (0.043 mole) and 70 ml of phosphorus oxychloride and the mixture stirred. To this, 11.3 g (0.04 mole) of 2-[2-(5-nitro-2-furyl)vinyl]-4-(3H)quinazolinone was added and rinsed into the flask with 15 ml of phosphorus oxychloride. The mixture was heated under reflux for 4 hours, cooled in an ice bath and diluted with 150 ml of diethyl ether. The 6-fluoro-2-[2-(5-nitro-2-furyl)vinyl]-4-chloroquinazoline which precipitated was collected by filtration, washed with 100-150 ml of diethyl ether, slurried in 100 ml of diethyl ether and then refiltered to obtain 8.09 g of the desired product.

### 3,4-dihydroxyaniline (V)

Concentrated hydrochloric acid (10 ml) was added to a mixture of 4-nitro-1,2-catechol (12 g) and tin (II) chloride (2 g) in ethanol (100 ml). The mixture was heated for 2 h and cooled to ambient temperature. The desired aniline was purified by flash chromatography.

### 2-[2-(5-nitro-2-furyl)vinyl]-4-(3,4-dihydroxyanilino)quinazoline (VI)

The following is the general procedure to obtain 4-[aminocatechol] derivatives of 2-[2-(5-nitro-2-furyl)vinyl]quinazoline. A round bottom flask equipped with a magnetic stirrer and oil bath for heating was charged with of 3,4-dihydroxyaniline (1 mole) and 3 ml of dimethylformamide. After the 3,4-dihydroxyaniline was dissolved by stirring, 2-[2-(5-nitro-2-furyl)vinyl]-4-chloroquinazoline (IV) (0.9 mmol) was added. The reaction mixture was then heated at 70° C.-90° C. for 2 hours after which 5 ml of water was added and the solution after cooling was placed in a refrigerator for crystallization. After 3 days, the brown yellow solid was collected, washed first with water, then methanol and then dried to obtain the desired product.

### EXAMPLE II

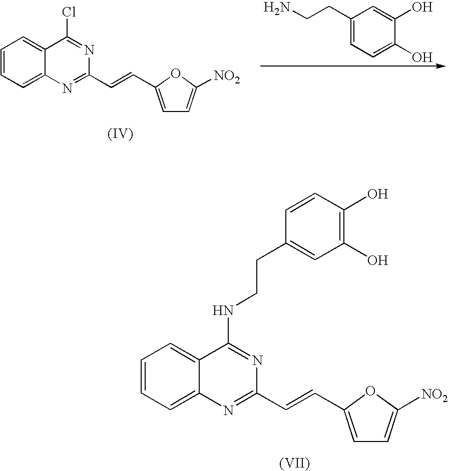

### 2-[2-(5-nitro-2-furyl)vinyl]-4-[2-(3,4-dihydroxyphenyl)ethylamino]quinazoline (VII)

This compound was prepared in the same manner as in Example I but by replacing 3,4-dihydroxyaniline (V) with 2-(3,4-dihydroxyphenyl)-ethylamine (1 mmol) to give the desired product as a solid after flash chromatography.

### EXAMPLE III

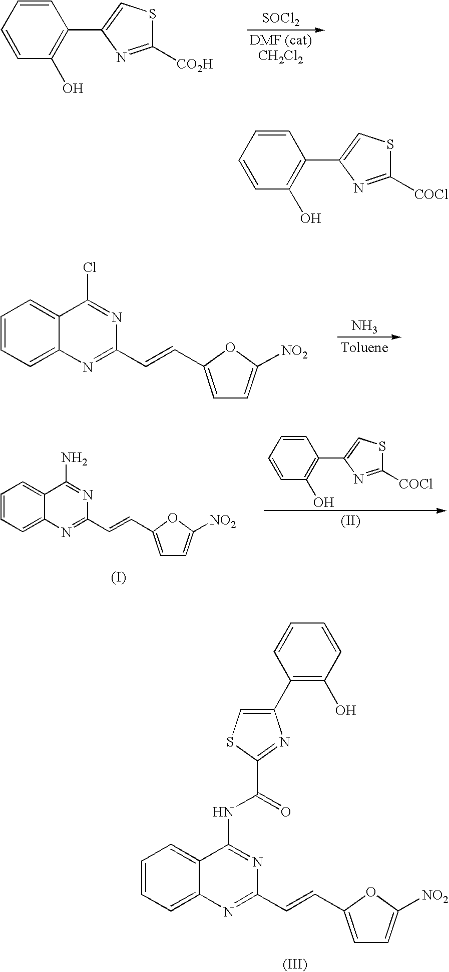

2-[2-(5-nitro-2-furyl)vinyl]-4-aminoquinazoline (I) 2M ammonia in toluene (5 mmol) was added to a solution of 2-[2-(5-nitro-2-furyl)vinyl]-4-chloroquinazoline (3 mmol) in toluene. The container was sealed and the mixture was heated to 80° C. for 4 h. The mixture was cooled and evaporated. The desired compound was purified by flash chromatography.

### 2-(2-hydroxyphenyl)-2-thiazole-4-carboxoyl chloride (II)

2-(2-hydroxyphenyl)-2-thiazole-4-carboxylic acid (3 mmol) obtained as described in U.S. Pat. No. 6,403,623 was treated with thionyl chloride (3.1 mmol) and a catalytic amount of dimethylformamide in dichloromethane (50 ml) at 0° C. The reaction was allowed to warm to ambient temperature for 2 h. The solvent was removed and the material dried under vacuum.

### 2-[2-(5-nitro-2-furyl)vinyl]-4-[2-(2-hydroxyphenyl)-2-thiazole-4-carboxylamido]quinazoline (III)

Dry dichloromethane (100 ml) was added to the product of (II) (1 mmol) and the mixture was cooled in an ice bath. The product of (I) was dissolved in pyridine (0.9 mmol in 20 ml) and added over 10 min to the dichloromethane solution. The reaction was allowed to stir 2 h at ambient temperature. The solvent was removed under reduced pressure. The desired product was purified by flash chromatography.

### EXAMPLE IV

### (S)-2-(2-hydroxyphenyl)-2-thiazoline-4-carboxoyl chloride (I)

(S)-2-(2-hydroxyphenyl)-2-thiazoline-4-carboxylic acid (3 mmol) obtained as described in U.S. Pat. No. 6,403,623 was treated with thionyl chloride (3.1 mmol) and a catalytic amount of dimethylformamide in dichloro-methane (50 ml) at 0° C. The reaction was allowed to warm to ambient temperature for 2 h. The solvent was removed and the material dried under vacuum.

### (S)-2-[2-(5-nitro-2-furyl)vinyl]-4-[2-(2-hydroxyphenyl)-2-thiazoline-4-carboxylamido]quinazoline (II)

This compound was obtained in the same manner as that described in Example III but using (S)-2-(2-hydroxy-phenyl)-2-thiazoline-4-carboxoyl chloride (1 mmol) with 2-[2-(5-nitro-2-furyl)vinyl]-4-aminoquinazoline (0.9 mmol). The product was purified by flash chromatography.

### EXAMPLE V

### (R)-2-(2-hydroxyphenyl)-2-thiazoline-4-carboxoyl chloride (I)

(R)-2-(2-hydroxyphenyl)-2-thiazoline-4-carboxylic acid (3 mmol) obtained as described in U.S. Pat. No. 6,403,623 was treated with thionyl chloride (3.1 mmol) and a catalytic amount of dimethylformamide in dichloro-methane (50 ml) at 0° C. The reaction was allowed to warm to ambient temperature for 2 h. The solvent was removed and the material dried under vacuum.

### (R)-2-[2-(5-nitro-2-furyl)vinyl]-4-[2-(2-hydroxyphenyl)-2-thiazoline-4-carboxylamido]quinazoline (II)

This compound was obtained in the same manner as that described in Example III but using (R)-2-(2-hydroxy-phenyl)-2-thiazoline-4-carboxoyl chloride (1 mmol) with 2-[2-(5-nitro-2-furyl)vinyl]-4-aminoquinazoline (0.9 mmol). The product was purified by flash chromatography.

### EXAMPLE VI

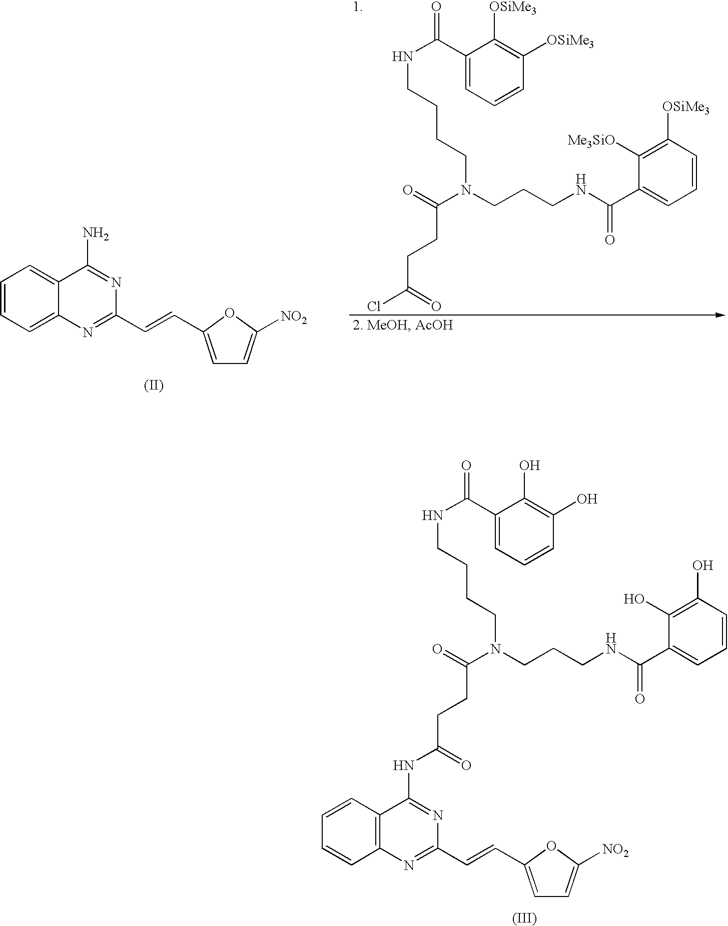

### 3-[1,10-bis(2,3-dihydroxybenzoyl)sperminylcarbonyl]propionic acid (I)

This compound was prepared in the same manner as described in Minnick, A. A., McKee, J. A., Dolence, E. K., Miller. M. J. Antimicrobial Agents and Chemotherapy, 1992, 35: 840-850, McKee, J. A., Sharma, S. K., Miller, M. J., Bioconjugate Chemistry, 1991, 2: 281-291.

### 2-[2-(5-nitro-2-furyl)vinyl]-4-{3-[1,10-bis(2,3-dihydroxy-benzoyl)sperminylcarbonyl}propionamido]quinazoline (III)

Bistrimethylsilyltrifluoroacetamide (100 μl) was added to a solution of compound I (0.1 mmol) in dry dichloromethane (1 ml) and the solution was stirred 2 h at ambient temperature. The solvent was removed to dryness and the product was placed under vacuum. The material was again dissolved in dichloromethane (1 ml) and cooled to 0° C. Oxalyl chloride (1.1 eq) was added and a catalytic amount of DMF. The reaction was continued for 40 min after the bubbling had stopped. A solution of 2-[2-(5-nitro-2-furyl)vinyl]-4-aminoquinazoline (II) (0.8 mmol) in pyridine (200 μl) and ether (200 μl) was added and the mixture was stirred overnight. The mixture was concentrated in vacuo and the residue was stirred in the presence of methanol with a catalytic amount of acetic acid for 2 h. After concentration, the product was purified by flash chromatography.

### EXAMPLE VII

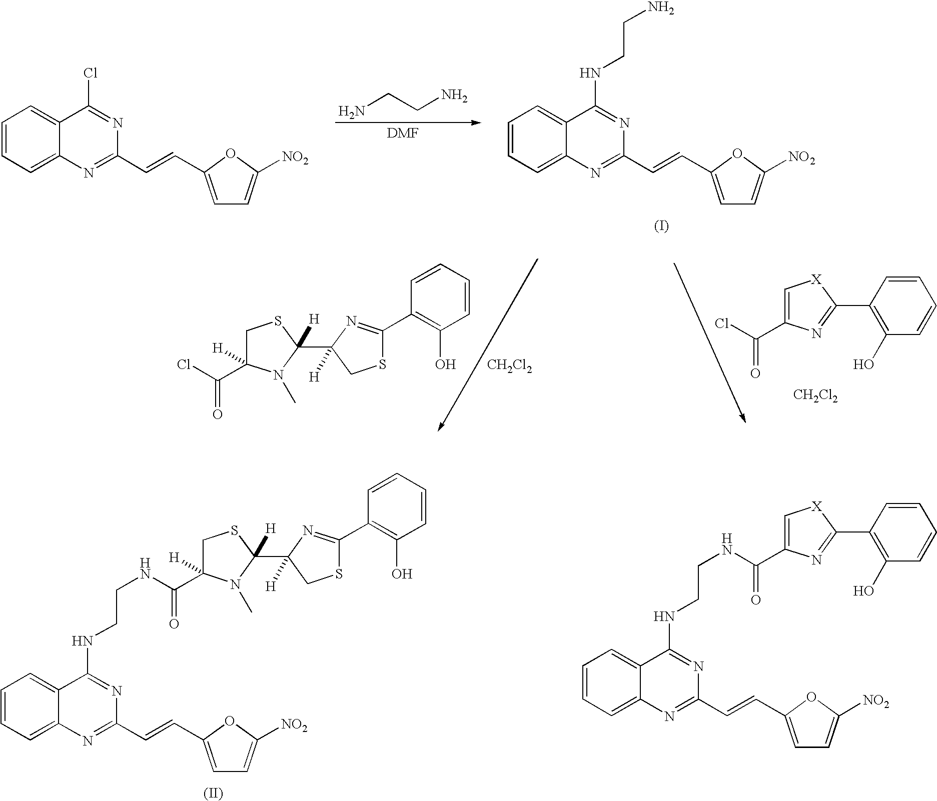

### 2-[2-(5-nitro-2-furyl)vinyl]-4-[2-aminoethylamino]quinazoline (I)

A solution of 1,2-diaminoethane (2 mmol) in toluene (1 ml) was added to a solution of 2-[2-(5-nitro-2-furyl)vinyl]-4-chloroquinazoline (1 mmol) in DMF (10 ml) under nitrogen atmosphere. The reaction vessel was sealed and heated to 80 degrees C. for 2 h. The mixture was cooled and the solvent was removed under vacuum. The residue was purified by flash chromatography to give the desired product.

### 2-[2-(5-nitro-2-furyl)vinyl]-4-[2-pyochelinylamidoethylamino]quinazoline (II)

Pyochelin (0.5 mmol) was treated with thionyl chloride (0.51 mmol) and a catalytic amount of dimethylformamide in dichloromethane (5 ml) at 0° C. The reaction was allowed to warm to ambient temperature for 2 h. The solvent was removed and the material dried under vacuum.

Dry dichloromethane (5 ml) was added to the acyl chloride prepared above and the mixture was cooled in an ice bath. Compound II of example III was dissolved in pyridine

(0.5 mmol in 2 ml) and added over 10 min to the dichloro-methane solution. The reaction was allowed to stir 2 h at ambient temperature. The solvent was removed under reduced pressure. The desired product was purified by flash chromatography.

Pyochelin analogue synthesis is described in the following reference: A. Zamri, I. J. Schalk, F. Pattus, M. A.

Abdallah. Bacterial Siderophores: Synthesis and Biological Activities of Novel Pyochelin Analogues. Bioorg. Med. Chem. Lett., 2003, 13, 1147-1150.

### EXAMPLE VIII

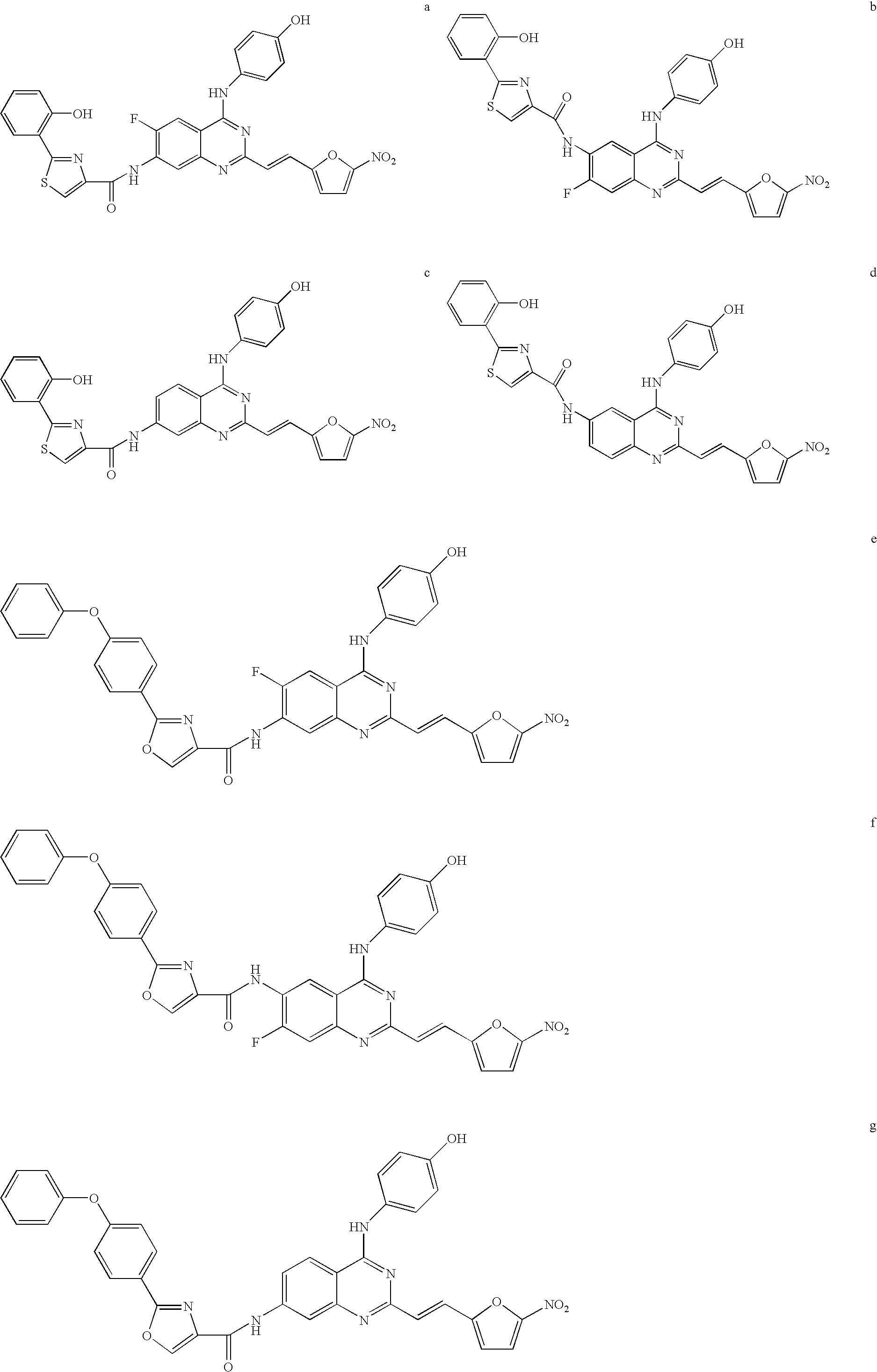

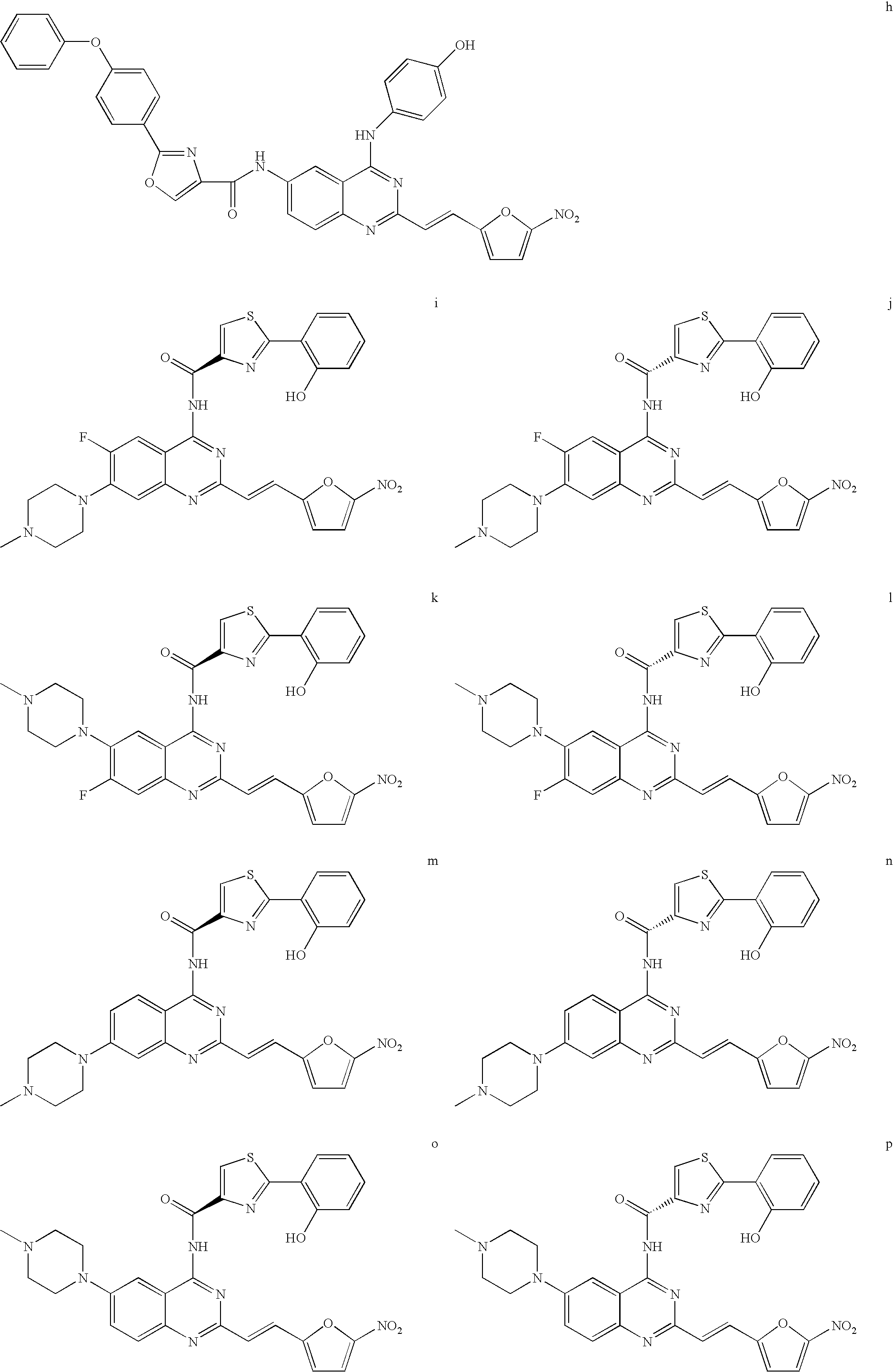

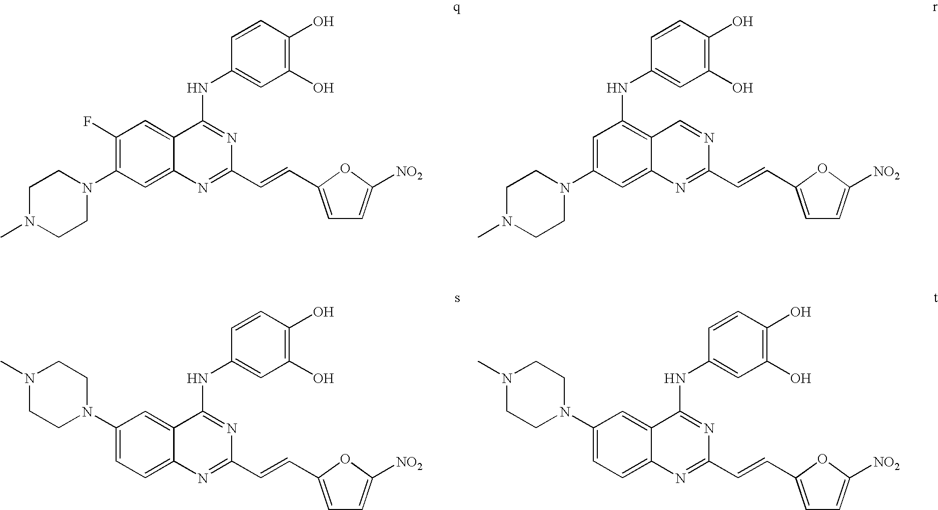

### EXAMPLE IX

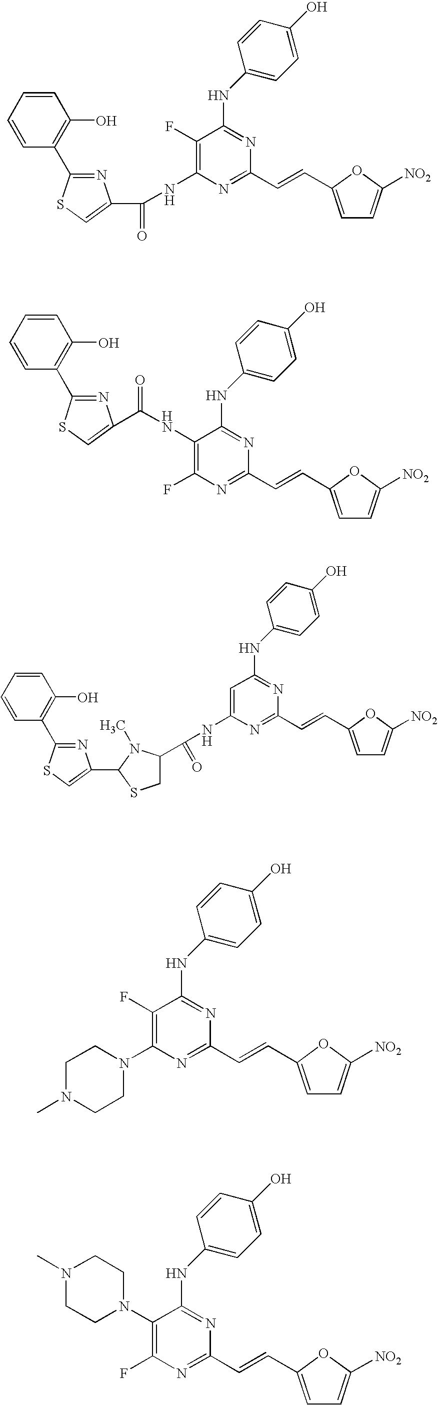

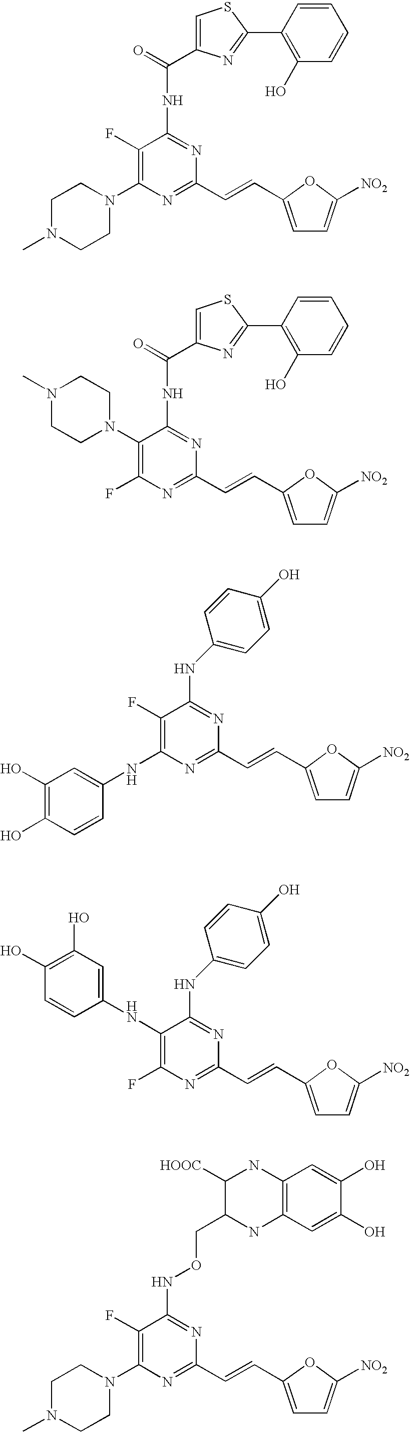

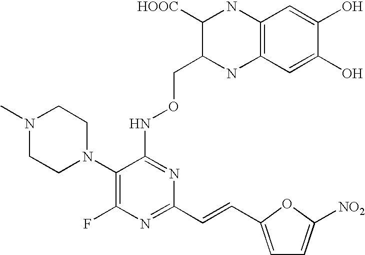

### EXAMPLE X

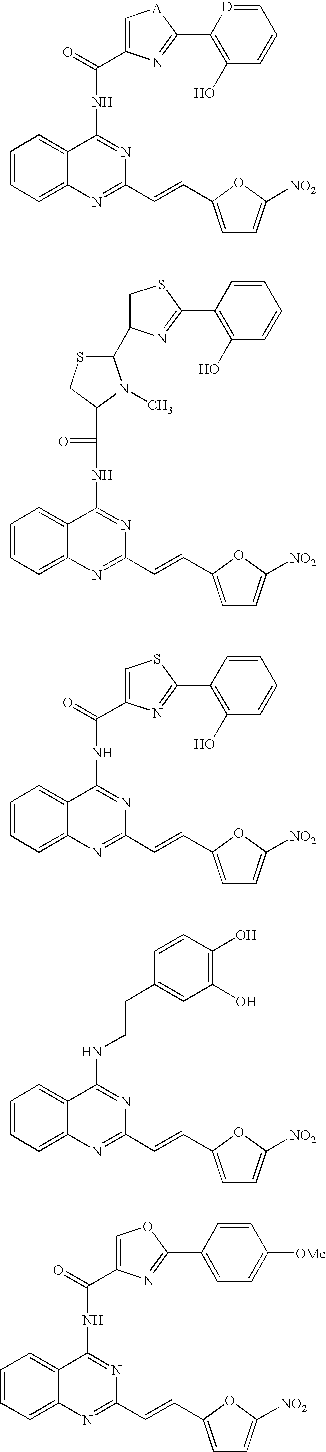

### EXAMPLE XI

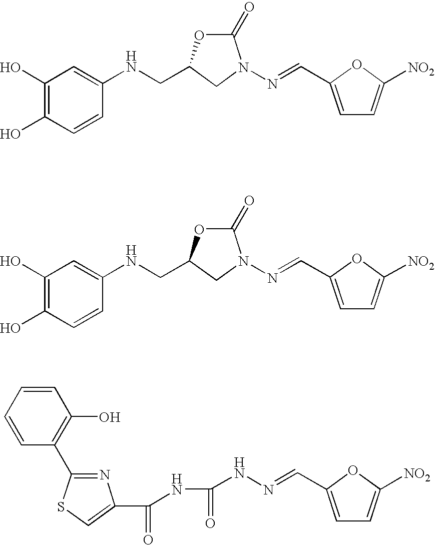

### EXAMPLE XII

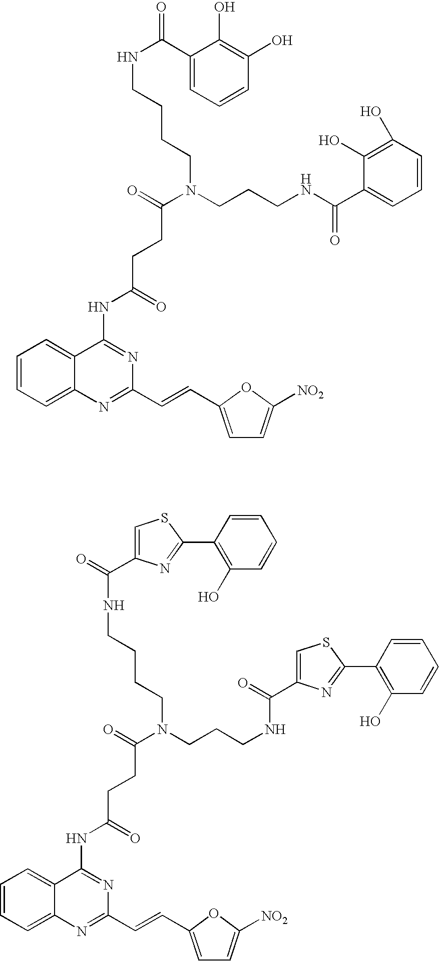

### EXAMPLE XIII

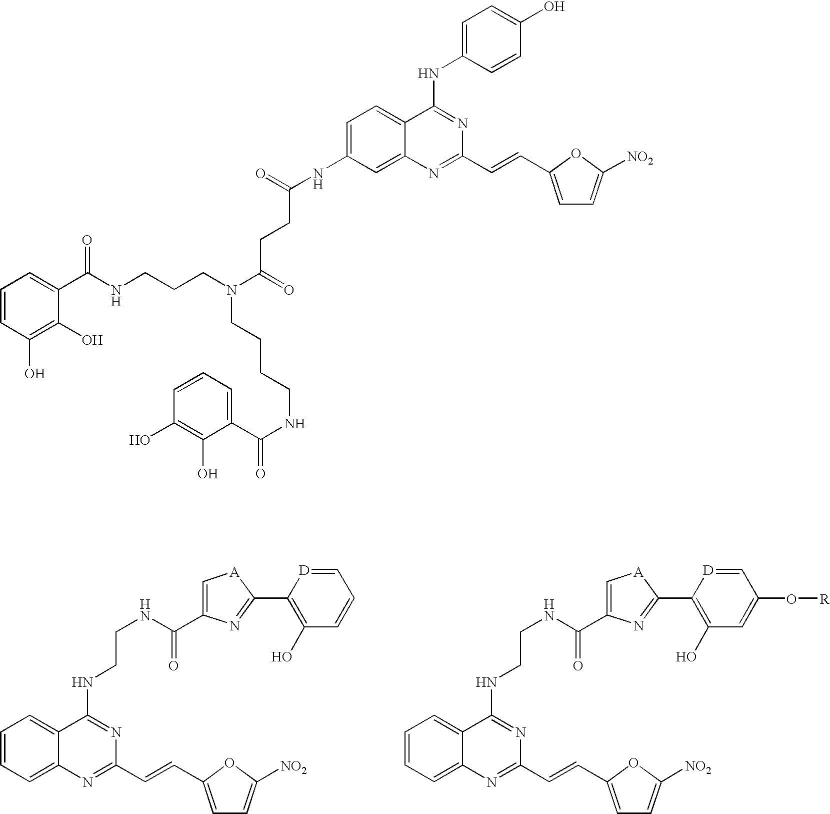

The prepared compounds were evaluated for antimicrobial activity by the following procedures.

### Minimal Inhibitory Concentration (MIC) Determination.

Bacteria. Susceptibility tests were performed against several bacterial species following the recommendations from the National Committee for Clinical Standards (NCCLS). Examples of microbial strains tested are provided in Table 1. The MICs were determined by a broth microdilution technique using a final volume of 100 μl of cation-adjusted Mueller Hinton Broth (MHBCA) and a bacterial inoculum of 10⁵-10⁶ Colony Forming Units (CFU)/ml. The inocula were verified and precisely determined by applying 10 μl drops of 10 fold dilutions onto Triptic Soy Agar plates. The CFU were counted after an incubation of 24 h at 35° C. Any experiment showing an inoculum that was more or less than 10⁵-10⁶ CFU/ml was rejected. Control antibiotics and test compounds were prepared at a concentration equivalent to 2 fold the highest desired final concentration. Compounds were then diluted directly in the 96-well microtiter plates by serial 2-fold dilutions using a multichannel pipette. Microtiter plates were incubated for 24 h at 35° C. and growth was recorded by using a microtiter plate reader at 650 nm as well as by visual observation. The MIC was defined as the lowest concentration of compound yielding no visible growth. At least two commercial antibiotics (e.g., imipenem and vancomycin) were always included as internal microtiter plate controls in each MIC assay. Results were rejected from any microtiter plate that showed a discrepancy in such control antibiotic MICs compared to the NCCLS reference data for ATCC strains (a MIC differing by more than 2 doubling dilutions).

Fastidious bacteria. The medium used for Streptococcus pneumoniae, L. monocytogenes, Neisseria meningitidis, and Campylobacter jejuni was MHBCA containing 2% laked horse blood. The medium used for Haemophilus influenzae and Branhamella (Moraxella) catarrhalis was HTM as recommended by the NCCLS. Cultures of these fastidious bacteria were incubated at 35° C. in a 5% CO₂ atmosphere. The medium used for Bacteroides fragilis was Wilkins Chalgren broth and growth was allowed under an anaerobic atmosphere at 35° C. for 48 hours. The MHBCA medium used to grow Mycobacterium smegmatis prior to the MIC assays was supplemented with 0.02% Tween-80 and results from microtiter plates were read after 48 hours of incubation. The medium used for Bacteroides fragilis was Wilkins Chalgren broth and growth was allowed under an anaerobic atmosphere at 35° C. for 48 hours.

Yeasts. Susceptibility tests for yeasts were also done accordingly to the NCCLS recommendations. The tests differed from those performed for bacteria in the following manner: (1) the medium recommended and used was RPMI for Candida albicans; (2) the inoculum used for yeasts was 0.5×10³ to 2.5×10³ CFU/ml; and (3) the incubation was of 48 h at 35° C. Also for yeasts, the microtiter plates were carefully vortexed after the incubation period and the MIC was defined as the lowest concentration of compound that caused a prominent decrease in turbidity (at least 80% of growth inhibition).

Time-kill curves. The bactericidal action of compounds was also evaluated over time (time-kill curve experiments). A bacterial inoculum of 1×10⁵-5×10⁵ Colony Forming Units (CFU)/ml was prepared. The inocula were verified and precisely determined by applying 10 μl drops of 10-fold dilutions onto Triptic Soy Agar plates. The CFU were counted after an incubation of 24 h at 35° C. Any experiment showing an inoculum that was more or less than the desired range of CFU/ml was rejected. Time-kill curve experiments were performed in 30 ml of MHB placed in 50 ml shaking flasks over a period of 24 hours. Test compounds and control antibiotics were added at time 0 hour and, at each time point, a sample was removed from flasks and the CFU determined by plate counts as described above. CFU from compound-treated cultures were compared to CFU collected from the control flask without antibiotic. Test compounds and control antibiotics were assayed at a concentration of 0.5MIC and at the MIC as determined by a broth microdilution technique as described above.

Mode of action studies. Macromolecular biosynthesis assays were performed to identify the microbial cellular processes selectively affected by antibiotic compounds. Exponentially growing bacteria in MHB were washed and diluted in a complete synthetic medium to an optical density of 0.2 (at 600 nm). Cells were then distributed in 96-well plates containing serially diluted antibiotic compounds and radiolabeled precursors. The radiolabeled precursors were D-[³H]-alanine for peptidoglycan synthesis, [³H]-thymidine for DNA synthesis, [³H]-uridine for RNA synthesis and [³H]-leucine for protein synthesis. Incorporation was allowed for 30 minutes at 35° C. before macromolecules were precipitated for 1 hour on ice in the presence of 10% trichloroacetic acid. The radioactive precipitates were then collected onto GC filters and the radioactivity measured by liquid scintillation counting. Data were expressed as the percentage of radioactivity incorporated compared to control cells grown in the absence of antibiotic compound.

In vivo efficacy. The antimicrobial activity of compounds was also evaluated in a S. aureus model of systemic infection in the mouse. To produce the systemic infection, CD-1 female mice (20 g) were injected intra-peritoneally with 10⁷ CFU of S. aureus strain Newman suspended in 0.5 ml of endotoxin-free PBS containing 5% mucin (w/v). The compounds were administrated at 1 hour post-infection and kidneys harvested and pooled, for each animal, 5 hours after bacterial inoculation. Tissues were homogenized in PBS and homogenates serially diluted and plated for CFU determination.

Compounds were evaluated against several microorganisms in order to determine their microbial growth inhibition activity and breadth of spectrum.

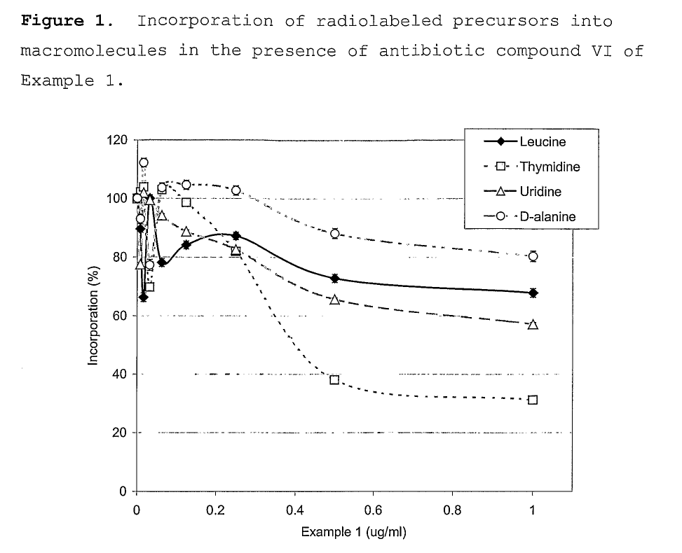

For example, compound VI of Example 1 is active against a wide variety of clinical isolates and reference strains of Gram positive and Gram negative bacteria. Compound VI of Example 1 was tested side-by-side with other antibiotics representative of various classes of compounds commercially available. Compound VI of Example 1 was potent against E. coli and its activity was superior to that of other nitrofurans (e.g. furazolidone, nitrofurantoin and nitrofurazone) (Table 2). Initial mode of action studies demonstrated that the antibiotic effect of the Example 1 compound VI may be produced through an inhibition of DNA metabolism, an essential cell process for microbes (see FIG. 1). This effect was similar to that observed for norfloxacin, a known inhibitor of DNA topoisomerase and DNA metabolism, and different from the observed effect of chloramphenicol (a protein synthesis inhibitor) and vancomycin, an inhibitor of cell wall peptidoglycan synthesis (data not shown). Compound VI of Example 1 was active in a S. aureus systemic infection model in the mouse. Results showed that the Example 1 compound VI reduced the presence of viable bacteria in the kidneys. This result demonstrated bioavailability of the Example 1 compound VI and its relatively low toxicity in vivo.

Compound “q” of Example VIII showed an antibacterial activity against bacteria generally causing severe opportunistic and/or nosocomial infections. These included Gram positive (Methicillin-Resistant and Methicillin-Sensitive S. aureus strains [MRSA and MSSA, respectively with a MIC of 4-8 ug/ml], and Enterococci, like E. faecalis with a MIC of 8 ug/ml) and Gram negative bacteria (including species causing difficult-to-treat infections like Yersinia enterocolytica and Burkholderia cepacia with a MIC of 8 ug/ml) and anaerobic bacteria (MIC of 8 ug/ml). The MIC (ug/ml) of compound “q” of Example VIII was better than that of traditional antibiotic classes, like beta-lactams, fluoroquinolones or macrolides, in bacterial strains that showed resistance mechanisms to these antibiotics (Table 3), as well as a greater antibacterial activity than that observed for nitrofurantoin, a traditional nitrofuran antibiotic lacking the novel structural features described in the present invention.

Compound “q” of Example VIII also showed growth inhibitory activity against typical respiratory tract pathogens causing community-acquired otitis media and pneumonia, like Haemophilus influenzae and Branhamella (Moraxella) catarrhalis (MIC of 1 ug/ml). In addition, compound “q” of Example VIII also showed inhibitory activity against the bacterial genus Mycobacterium (MIC of 8 ug/ml). The bacterium Mycobacterium tuberculosis, that is one of the etiologic agents causing tuberculosis, is also a member of that bacterial genus. Besides, compound “q” of Example VIII also demonstrated a very good activity (MIC of 8 ug/ml) against three species of the bacterial genus Bacillus (i.e., B. cereus, B. subtilis and B. atrophaeus). Bacillus anthracis, the bacterial pathogen causing anthrax, is also a member of that bacterial genus.

TABLE 1
Examples of microbial species used in the evaluation
of antimicrobial activity of compounds.
Primary Strain Panel:

Gram positive.
Staphylococcus aureus ATCC 29213
Staphylococcus aureus MRSA COL
Staphylococcus epidermidis ATCC 12228
Staphylococcus saprophyticus ATCC 15305
Enterococcus faecalis ATCC 29212
Enterococcus faecium ATCC 35667
Bacillus cereus ATCC 11778
Bacillus subtilis ATCC 6633
Bacillus atrophaeus ATCC 9372
Streptococcus pneumoniae ATCC 49619
Listeria monocytogenes ATCC 13932
Gram negative.
Escherichia coli ATCC 25922
Citrobacter freundii ATCC 8090
Klebsiella oxytoca ATCC 43165
Klebsiella pneumoniae ATCC 13883
Enterobacter aerogenes ATCC 35029
Enterobacter cloacae ATCC 35030
Proteus mirabilis ATCC 25933
Serratia marcescens ATCC 8100
Pseudomonas aeruginosa ATCC 27283
Acinetobacter baumanii ATCC 10606
Burkholderia cepacia ATCC 27515
Yersinia enterocolytica ATCC 23715
Haemophilus influenzae ATCC 49247
Haemophilus influenzae ATCC 49766
Branhamella (Moraxella) catarrhalis ATCC 8176
Neisseria meningitidis ATCC 13102
Campylobacter jejuni ATCC 33291
Anaerobic bacteria.
Bacteroides fragilis ATCC 25285
Yeasts and fungi.
Candida albicans ATCC 10231

TABLE 2
MICs in ug/ml for control antibiotics and the
compound VI of Example 1 obtained for E. coli.

| | E. coli |
|---|---|
| Antibiotic | ATCC 25922 |
| Example 1, Compound VI | 0.5 |
| Ampicillin | 4-8 |
| Cefotaxime | 0.06-0.12 |
| Ceftriaxone | 0.03-0.06 |
| Chloramphenicol | 4-8 |
| Erythromycin | 64 |
| Furazolidone | 1-2 |
| Gentamicin | 0.5-2 |
| Imipenem | 0.12 |
| Meropenem | 0.015-0.06 |
| Nitrofurantoin | 8-16 |
| Nitrofurazone | 8-16 |
| Norfloxacin | 0.03-0.06 |
| Oxacillin | 512->512 |
| Rifampicin | 8 |
| TMP/SMX (1/19) | 0.25/4.75-0.5/9.5 |
| Vancomycin | >64 |

TABLE 3
MICs in μg/ml for control antibiotics and Examples VIII Compound “q” obtained
for a variety of antibiotic multi-resistant MRSA strains.

| | Example VIII | | | | | |
|---|---|---|---|---|---|---|
| MRSA strains | compound “q” | Oxacillin | Erythromycin | Norfloxacin | Gentamicin | Nitrofurantoin |
| Sa211c | 8 | 16-32 | >32 | >32 | 1 | 16-32 |
| Sa212c | 8 | 16 | >32 | >32 | 0.25 | 32 |
| Sa220c | 4 | 8-16 | 0.5 | >32 | 0.5 | 16 |
| Sa224c | 8 | 32-64 | >32 | >32 | 0.25 | 16 |
| Sa228c | 8 | 128 | >32 | >32 | 32 | 16-32 |
| Sa234c | 8 | 32−>128 | >32 | >32 | 1 | 16-32 |
| Sa248c | 8 | 512 | >128 | 32 | >128 | 16 |
| Sa249c | 8 | 512 | >128 | 32 | >128 | 16 |
| Sa253c | 8 | 1024 | >128 | >128 | >128 | 16 |

1. A compound of the formula (1.0) 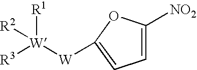
wherein
W is absent, vinyl (cis or trans —CH═CH—, preferably trans) or —N═CH—;
W′ is absent, or, shown with R¹, R² and R³, is 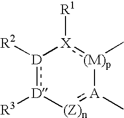
(central structure)
wherein the dotted line together with the solid line represent either a single or a double bond, D, D″, X, M and Z are each independently selected from CH, C, O, S, NH and N and A is selected from C, N or P, however at least one of D or X is C, and preferably no sore than two D, X, M, A or Z are O, S, NH or N, and D″ must be C or CH, unless R² and R³ are taken together to form a ring for example quinazoline; and p and n are each independently selected from 0, 1, and 2;
R¹, R² and R³ are each independently selected from absent, hydrogen, halogen (includes halogen atoms fluorine, chlorine, bromine and iodine), CH₃, C₁-C₁₀ alkyl, C₂-C₁₀ alkenyl, C₂-C₁₀ alkynyl, C₁-C₁₀ alkoxy, C₂-C₁₀ alkenyloxy, C₂-C₁₀ alkynyloxy, aryl, OH, trifluoromethyl, methylenedioxy, phenoxy, OR⁴, CO₂R⁴, SO₂R⁴, PO(OR⁴)₂, CON(R⁴)₂, OAr, NH₂, NHR⁴, NR⁴, N(R⁴)₂, NHAr, SH, SR⁴, SAr, ═O, hydroxamic acid, heterocyclic ring, a solubilizing group as defined below, and a VQT moiety (the distal ring system) as defined below with the proviso that VQT is present at least once, wherein the alkyl, alkenyl, alkynyl, alkoxy, alkenyloxy or alkynyloxy group may be unsubstituted or substituted, preferably, with 1-5 halogen atoms or 1-2 OR⁴ groups, and aryl is, preferably, selected from the group consisting of: phenyl, naphthyl, indolyl, biphenyl, phenoxyphenyl, pyridyl, furanyl, thiophenyl and bithienyl, said aryl group being optionally substituted, preferably, by 1-3 groups selected from R⁴, a solubilizing group as defined below, and a VQT moiety as defined below;
the solubilizing group can be, without limitation, preferably 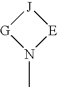
wherein G and E are each independently selected from CH₂, CH₂CH₂ and CH₂CH-alkyl; and J is O, NH or NCH₃,
R⁴ is selected from CH₃, C₁-C₁₀ alkyl, C₂-C₁₀ alkenyl, C₂-C₁₀ alkynyl, C₁-C₁₀ alkoxy, C₂-C₁₀ alkenyloxy, C₂-C₁₀ alkynyloxy, aryl, heterocyclic ring, a solubilizing group as defined above, and aryl is, preferably, selected from the group consisting of; phenyl, naphthyl, indolyl, biphenyl, a substituted or unsubstituted mono or bi carbo- or hetero-cyclic structure having 4-14 atoms which are aliphatic or aromatic in nature and any combinations with a substituted or unsubstituted mono or bi carbo- or hetero-cyclic aliphatic or aromatic structure having 3 to 8 atoms;
V is C, CH, N, NH, O or 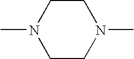
Q acts as a stable or labile linker, for example selected from absent, C₁-C₁₀ alkyl, C₂-C₁₀ alkenyl, C₂-C₁₀ alkynyl, C₁-C₁₀ amine, C₂-C₁₀ alkoxy, C₂-C₁₀ alkenyloxy, C₂-C₁₀, alkynyloxy, phenyl, heterocyclic ring, —C(═O)—, —SO₂—, —PO(OR¹)—, —NOH, —(CH₂)ₜ—C(═O)—, and —(CH₂)ₜ—NH—C(═O)— wherein t is 0 to 10; and
T is 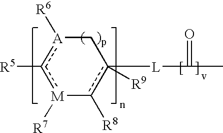
wherein A and M are independently selected from C, CH, O, NH, N and S; p, n and v are each independently selected from 0 and 1, preferably n ˜1 and p=0; and the dotted line together with the solid line represent either a single or a double bond; and
R⁵, R⁶, R⁷, R⁸ and R⁹ are each independently selected from absent or as defined for R¹;
the preferred R⁵ is: 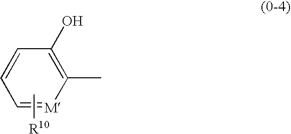
wherein M′ is independently selected from CH, C and N;
and wherein R¹⁰ is absent or as defined for R¹;
L is acting as a stable or labile linker, for example selected from absent, C₁ to C₁₀ alkyl, C₂ to C₃₀ alkenyl, C₂ to C₁₀ alkynyl, C₁-C₁₀ amine, C₁-C₁₀ alkoxy, C₂-C₁₀ alkenyloxy, C₂-C₁₀ alkynyloxy, phenyl, —C(═O)—, —PO(OR¹)—, —NOH—, —(CH₂)ₜ—C(═O)—, and —(CH₂)ₜ—NH—C(═O)— wherein t is 0 to 10, and a heterocyclic ring, wherein the heterocyclic ring is preferably 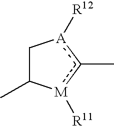
wherein A and M are independently selected from C, CH, N, NH, O, or S; and wherein R¹¹ and R¹² are independently selected from absent, or as defined for R¹; and wherein the dotted line together with the solid line represent either a single or a double bond;
when n=0 in T. at least one of R⁵, R⁶, R⁷, R⁸ or R⁹ must be either a catechol as shown: 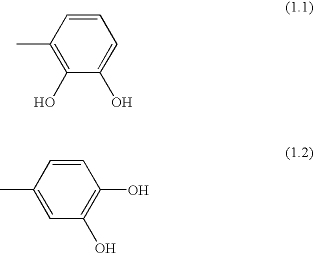
or a hydroxamic acid (R¹═H) or hydroxamate (R¹ as described above) as shown: 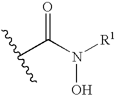
R² and R³, when taken together in formula 1.0, (wherein W′ is the central structure), form in combination with the D and D″ atoms of the central structure to which they are fused, the following: 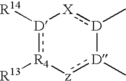
wherein the dotted line together with the solid line represent either a single or a double bond, X, D, D′, D″, D′″, Z are each independently selected from CH, C, O, S, NH, and N; and n is selected from 0, 1 and 2;
wherein R¹³ and R¹⁴ are each independently as defined for R¹ or taken together to form a ring such as for example a quinazoline;
and when R² and R³ are taken together in formula 1.0,
R¹ is as defined above, or 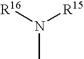
wherein R¹⁵ and R¹⁶ are each independently as defined for R⁴;
with the proviso that there must be present in the compound of formula 1.0 at one, two or three positions at least one VQT moiety which is selected from one or more of: 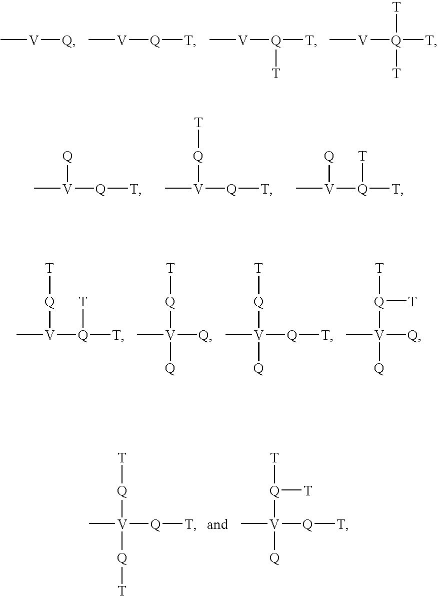
or enantiomers thereof;
wherein the compound of formula 1.0 has from 1 to 9 T, and each V and Q and T is selected independently;
or a pharmaceutically acceptable salt of such compound.

2. A compound of the formula 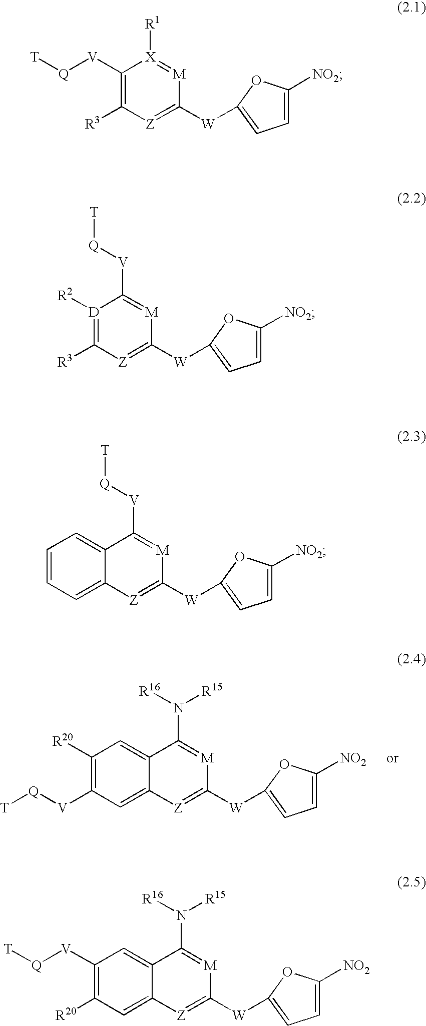
wherein
w is absent or is vinyl (cis or trans —CH═CH—, preferably trans);
D, X, M and Z are each independently selected from CH, C and N;
V is NH, O, S or 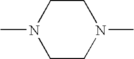
Q acts as a stable or labile linker, for example selected from absent, C₁ to C₁₀ alkyl, C₂ to C₁₀ alkenyl, C₂ to C₁₀ alkynyl, C₁-C₁₀ amine, C₁-C₁₀ alkoxy, C₂-C₁₀ alkenyloxy, C₂-C₁₀ alkynyloxy, phenyl, heterocyclic ring, —C(═O)—, —PO(OR¹)—, —SO₂—, —NOH, —(CH₂)ₜ—C(═O)—, and —(CH₂)ₜ—NH—C (═O)— wherein t is 0 to 10;
T is selected from: 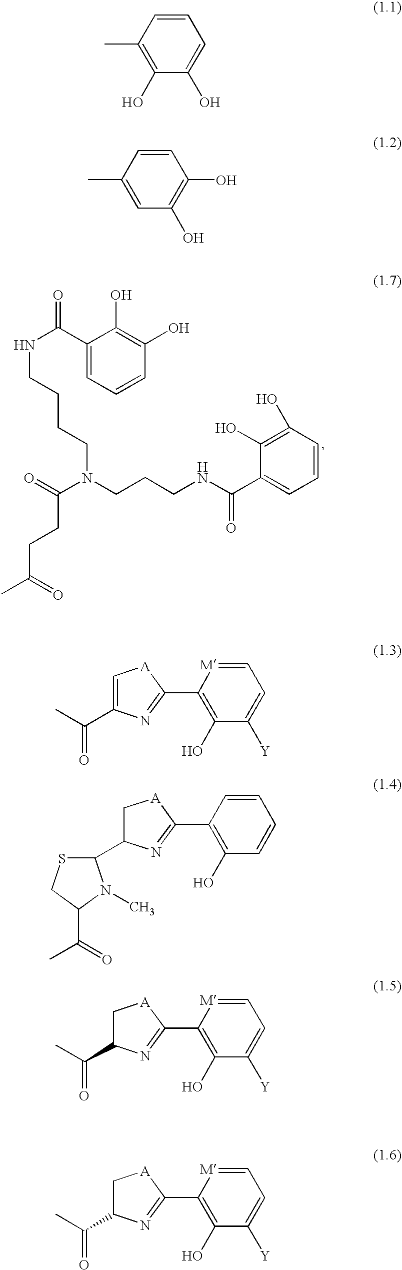
wherein A is selected from O, NH, NR and S; M′ is CH or N; and Y is H or OH;
R³ is hydrogen, alkyl, aryl, OH, OR, OAr, NH₂, NHR, NHAr, NHAr, SH, SR, or SAr; and
R¹ and R² are each independently selected from:
absent (when the X to which R₁ or R₂ is bonded is N or CH), hydrogen, alkyl, aryl, OH, OR, OAr, NH₂, NHR, NHAr, SH, SR, and SAr;
R¹⁵ is selected from H, alkyl and substituted alkyl;
R¹⁶ is selected from alkyl, substituted alkyl, phenyl and substituted phenyl;
R²⁰ is halogen, a solubilizing group as defined in claim 1 or as defined for R³;
or a pharmaceutically acceptable salt of such compound.

3. The compound according to claim 1, wherein R² and R³ are taken together to form 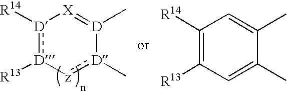
wherein the dotted line together with the solid line represent either a single or a double bond, X, D, D′, D″, D′″, Z are each independently selected from CH, C, O, S, NH, and N; and n is selected from 0, 1 and 2; and wherein R¹³ and R¹⁴ are each independently as defined for R¹.

4. The compound according to claim 2, wherein R²⁰ is a halogen or a solubilizing group as defined in claim 1.

5. A composition comprising the compound according to any one of claims 1 to 4 and a carrier, diluent or excipient.

6. A pharmaceutical composition comprising the compound according to any one of claims 1 to 4 and a pharmaceutically acceptable carrier.

7. A method for treating a bacterial infection in a human, comprising administering to said human a therapeutically effective amount of the compound according to any one of claims 1 to 4.

8. A method for preventing a bacterial infection in a human, comprising administering to said human a prophylactically effective amount of the compound according to any one of claims 1 to 4.

9. A method for disinfecting a surface of an object, including a human, of bacteria, which comprises: selecting an area of the surface for disinfection and applying the compound according to any one of claims 1 to 4 onto the surface of the object in an amount and for a time sufficient to achieve a desired degree of disinfection.

10. A method for sterilizing a surface of an object, including a human, of bacteria, which comprises; selecting an area of the surface for sterilization and applying the compound according to any one of claims 1 to 4 onto the surface of the object in an amount and for a time sufficient to achieve sterilization.

11. Use of the compound according to any one of claims 1 to 4 for treating or preventing bacterial infection.

12. Use of the compound according to any one of claims 1 to 4 in the manufacture of a medicament for treating or preventing bacterial infection.

13. Use of the compound according to any one of claims 1 to 4 for disinfection.

14. Use of the compound according to any one of claims 1 to 4 for antisepsis.

15. Use of the compound according to any one of claims 1 to 4 for sterilization.

16. The compound of claim 1 wherein V is C, CH, N, NH or O.

17. The compound of claim 2 wherein V is NH or O.
